(19) United States
(12) Patent Application Publication
(10) Pub. No.: US 2024/0306951 A1
(43) Pub. Date: Sep. 19, 2024

(54) SYSTEM AND METHOD FOR MONITORING A HEALTH PARAMETER OF A PERSON

(71) Applicant: KNOW LABS, INC., Seattle, WA (US)

(72) Inventor: JOHN CRONIN, Seattle, WA (US)

(21) Appl. No.: 18/603,882

(22) Filed: Mar. 13, 2024

Related U.S. Application Data
(60) Provisional application No. 63/452,782, filed on Mar. 17, 2023.

(51) Int. Cl. A61B 5/145; A61B 5/00; A61B 5/05

(52) U.S. Cl. CPC A61B 5/14532; A61B 5/05; A61B 5/681

(57) ABSTRACT

The present disclosure provides a method for monitoring a health parameter of a person in which a system receives data that corresponds to a digital pulse wave signal that is generated from radio frequency data that corresponds to radio waves that have responded from below the skin surface of a person, wherein the radio frequency data is collected through a three-dimensional array of receive antennas, the receive antennas are located at various locations to the transmit antennas, and determines a value that corresponds to a blood glucose level in the person in response to the data that corresponds to the digital pulse wave signal.

### FIELD

The present disclosure is generally related to monitoring a health parameter of a person.

### BACKGROUND

Diabetes is a medical condition in which a person's blood glucose level, also known as blood sugar level, is persistently elevated. Diabetes can result in severe medical complications, including cardiovascular disease, kidney disease, stroke, foot ulcers, and eye damage if left untreated. Typically, diabetes is caused by either insufficient insulin production by the pancreas, referred to as “Type 1 diabetes,” or improper insulin response by the body's cells referred to as “Type 2 diabetes.” Further, monitoring a person's blood glucose level and administering insulin when a person's blood glucose level is too high to reach the desired level may be part of managing diabetes. Depending on many factors, such as the severity of diabetes and the individual's medical history, a person may need to measure their blood glucose level up to ten times per day. Each year, billions of dollars are spent on equipment and supplies for monitoring blood glucose levels.

Moreover, regular glucose monitoring is a crucial component of diabetes care. Further, measuring blood glucose is generally an invasive procedure by giving a blood sample at a clinic or hospital. Home glucose monitoring is also possible using a variety of devices. The blood sample is obtained by pricking the skin using a tiny instrument. A glucose meter or glucometer is a tiny instrument that measures the sugar in the blood sample. The majority of glucose monitoring methods and devices require a blood sample.

Currently, available glucose monitoring devices also require a blood sample, usually by pricking a needle under the skin and then using a polling technique to determine the glucose level of a patient. These monitoring devices are almost 95 percent accurate and are also preferable by urban citizens. However, such monitoring devices are often prone to contamination as the patient may not be in sanitary conditions to give the blood sample. Currently, for noninvasive devices it is difficult to achieve a consistent radio frequency responded reading based upon various wrist anatomies. Also, it is problematic for noninvasive devices to achieve a lower signal to noise ratio in the receiving antennas based on the wrist anatomy. Lastly, it is a challenge for noninvasive devices to receive a signal that is not positionally dependent.

Thus, there is a need in the prior art to monitor a health parameter of a person.

### SUMMARY

Systems and methods for monitoring a health parameter of a person. In monitoring a health parameter of a person, a system receives data that corresponds to a digital pulse wave signal that is generated from radio frequency data that corresponds to radio waves that have responded from below the skin surface of a person, wherein the radio frequency data is collected through a three-dimensional array of receive antennas, the receive antennas are located at various locations to the transmit antennas, and determines a value that corresponds to a blood glucose level in the person in response to the data that corresponds to the digital pulse wave signal.

### DESCRIPTIONS OF THE DRAWINGS

FIG. 1: Illustrates a device for monitoring a health parameter of a person, according to an embodiment.

FIG. 2: Illustrates a illustrate a cross-sectional view of a wrist with ulna bone and the basilic vein, according to an embodiment.

FIG. 3: Illustrates a functional block diagram of a prior art sensor system.

FIG. 4: Illustrates a prior art example of the physical layout of circuit components on a semiconductor substrate.

FIG. 5: Illustrates a prior art IC device overlaid on the hand/wrist.

FIG. 6: Illustrates a prior art IC device overlaid on the back of the smartwatch.

FIGS. 7A and 7B: Illustrate a device integrated in a wrist band, according to an embodiment.

FIG. 8: Illustrates operation of a base operation module, according to an embodiment.

FIG. 9: Illustrates operation of a sweep TX RX module, according to an embodiment.

FIG. 10: Illustrates operation of a cross sweep TX RX module, according to an embodiment.

FIG. 11: Illustrates operation of a best sweep data extraction module, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

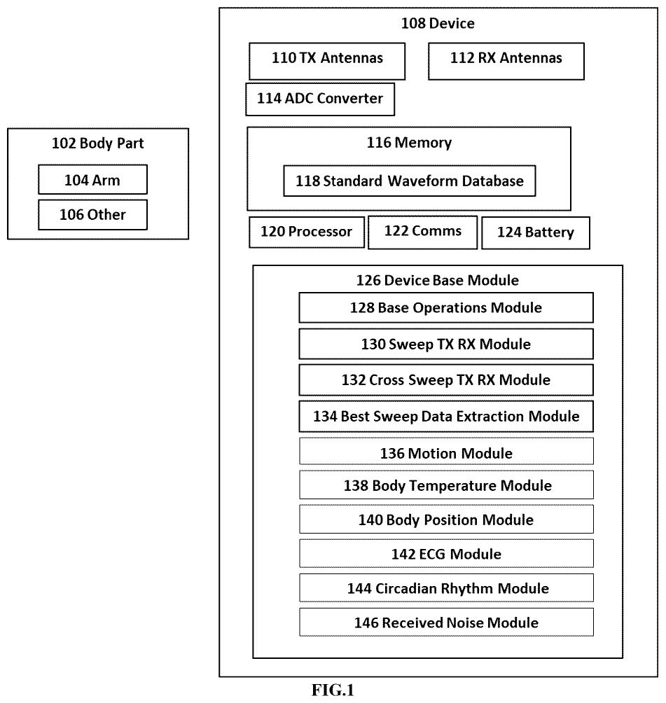

FIG. 1 illustrates a device for monitoring a health parameter of a person. This person has a body part 102 such as any part of a human including a limb or extremity. Further, embodiments may include the user having an arm 104 such as each of the two upper limbs of the human body from the shoulder to the hand. Further, embodiments may include other 106 parts of the body such as a limb or extremity, such as a leg or torso.

Further, the device 108 may be communicatively coupled to a device network. In one embodiment, the device network may be a wireless and/or wired communication channel. The device 108 may be worn by the user. The device 108 may determine health parameters using radio frequency signals in the RF Activated range. In one embodiment, the health parameters may include sugar or glucose levels. In embodiments, the system may target specific blood vessels using the RF Activated range radio frequency signals, which may output signals, and the output signals may correspond to the blood glucose level in the user. In one embodiment, the system may include integrated circuit (IC) devices (not shown) with transmit and/or receive antennas integrated in addition to that. Monitoring the blood glucose level in the specific blood vessels of the user using the RF Activated range radio frequency signals involves the transmission of suitable RF Activated range radio frequency signals below the user's skin surface. The RF Activated Range is defined by frequencies between 500 MHZ and 300 GHZ. Corresponding to the transmission, a responded portion of the RF Activated range radio frequency signals is received on multiple receive antennas. In an embodiment, the system isolates and/or processes a signal from a particular location of the blood vessels in response to the received RF Activated range radio frequency signals. The system may output a signal from the received RF Activated range radio frequency signals that correspond to the blood glucose level in the user. It can be noted that the device 108 may be worn by the user at various locations such as wrist, arm, leg, etc.

In one embodiment, the system for monitoring the blood glucose level of the user using the RF Activated range radio frequency signals involves transmitting RF Activated range radio frequency signals below the skin surface, receiving a responded portion of the RF Activated range radio frequency signals on multiple receive antennas, isolating a signal from the RF Activated range radio frequency signals at a particular location in response to the received RF Activated range radio frequency signals, and outputting a signal that corresponds to the blood glucose level in the user in response to the isolated signal.

In one embodiment, beamforming is used in the receiving process to isolate the RF Activated range radio frequency signals responded from a specific location on a specific blood vessel to provide a high-quality signal corresponding to the blood glucose levels in the specific blood vessel.

In another embodiment, Doppler effect processing may be used to isolate the RF Activated range radio frequency signals responded from the specific blood vessel's specific location to provide the high-quality signal corresponding to the blood glucose levels in the specific blood vessel.

It can be noted that analog and/or digital signal processing techniques may be used to implement beamforming and/or Doppler effect processing and digital signal processing of the received signals to dynamically adjust a received beam onto the desired location.

In another embodiment correlations between RX antennas and ground truth data may be used. In another embodiment, the beamforming and the Doppler effect processing may be used together to isolate the RF Activated range radio frequency signals responded from the specific location in the specific blood vessel to provide the high-quality signal corresponding to the blood glucose levels in the specific blood vessel.

In one exemplary embodiment, signals within the RF Activated range radio frequency signals of a higher frequency range of 122-126 gigahertz (GHz) having a shallower penetration depth are used to monitor blood glucose levels. It can be noted that the shallower penetration depth reduces undesirable reflections, such as reflections from bone and dense tissue such as tendons, ligaments, and muscle, which may reduce the signal processing burden and improve the quality of the desired signal that is generated from the location of the blood vessel. It can also be noted that bones are dielectric and semi-conductive. In addition, bones are anisotropic, so not only are bones conductive, but they also conduct differently depending on the direction of the flow of current through the bone. Alternatively, the bones are also piezoelectric materials. Therefore, signals within the Activated radio frequency signals of higher frequency range of 122-126 GHz in the shallower penetration depth are required to monitor the blood glucose levels.

Further, the device 108 may comprise one or more transmission TX antennas 110, one or more receiving RX antennas 112, an analog to digital converter (ADC) 114, a memory 116, a processor 120, a communication module 122 and a battery 124. The TX antennas 110 and/or the RX antennas 112 can be provided in a three-dimensional (3D) array, where the antennas 110 and/or 112 of the array are distributed such that they are not planar with one another. For example, the three-dimensional array may be a multi-dimensional array or an array of arrays, to increase the illumination pattern provided by the device 108. The TX antennas 110 and RX antennas 112 may be structured in three dimensional array which may be an array that has three dimensions, for example length, width, and height. For example, the 3D array may allow the TX antenna 110 and RX antenna 112 to provide coverage in multiple directions, including up and down, which allows the TX antenna 110 to send and the RX antenna 112 to receive signals from multiple angles. The 3D array may be designed to have TX antennas 110 and RX antennas 112 to be spatially dispersed throughout the device 108 to facilitate diversity reception, which improves the reliability and quality of the signal by reducing fading, interference, and multipath effects. The 3d array of the TX antennas 110 and RX antennas 112 allows the ability to capture weaker signals or transmit stronger signals.

In one embodiment, the device 108 may be a wearable and portable device such as, but not limited to, a cell phone, a smartwatch, a tracker, a wearable monitor, a wristband, and a personal blood monitoring device.

The one or more TX antennas 110 and the one or more RX antennas 112 may be fabricated over a substrate (not shown) within the device 108 in a suitable configuration. In one exemplary embodiment, at least two TX antennas 110 and at least four RX antennas 112 are fabricated over the substrate. The one or more TX antennas 110 and the one or more RX antennas 112 may correspond to a circuitry arrangement (not shown) over the substrate. Further, the ADC 114, the memory 116, the processor 120, the communication module 122, and the battery 124 may be fabricated over the substrate.

Further, the communication module 122 may be configured to facilitate communication between the device 108 and the device network. Further, embodiments may include a plurality of TX antennas 110 and a plurality of RX antennas 112. The one or more TX antennas 110 and the one or more RX antennas 112 may be integrated into the circuitry arrangement. The one or more TX antennas 110 may be configured to transmit the RF Activated range radio frequency signals at a pre-defined frequency. In one embodiment, the pre-defined frequency may correspond to a range suitable for the human body. For example, the one or more TX antennas 110 transmit signals within the RF Activated radio frequency signals at a range of 120-126 GHz. Successively, the one or more RX antennas 112 may be configured to receive the responded portion of the RF Activated range radio frequency signals. In one embodiment, the RF Activated range radio frequency signals may be transmitted to the user's skin, and electromagnetic energy may be responded from many parts such as fibrous tissue, muscle, tendons, bones, and the skin. It can be noted that effective monitoring of the blood glucose level is facilitated by an electromagnetic response of blood molecules, such as pancreatic endocrine hormones, against the transmitted RF Activated range radio frequency signals. It will be apparent to a skilled person that the pancreatic endocrine hormones such as insulin and glucagon are responsible for maintaining sugar or glucose level. Further, the electromagnetic energy responded from the blood molecules may be received by the one or more RX antennas 112.

Further, embodiments may include an ADC converter 114 which may be coupled to the one or more RX antennas 112. The one or more RX antennas 112 may be configured to receive the responded RF Activated range radio frequency signals. The ADC 114 may be configured to convert the RF Activated range radio frequency signals from an analog signal into a digital processor readable format.

Further, embodiments may include a memory 116 may be configured to store the transmitted RF Activated range radio frequency signals by the one or more TX antennas 110 and receive a responded portion of the transmitted RF Activated range radio frequency signals from the one or more RX antennas 112. Further, the memory 116 may also store the converted digital processor readable format by the ADC 114. In one embodiment, the memory 116 may include suitable logic, circuitry, and/or interfaces that may be configured to store a machine code and/or a computer program with at least one code section executable by the processor 120. Examples of implementation of the memory 116 may include, but are not limited to, Random Access Memory (RAM), Read Only Memory (ROM), Hard Disk Drive (HDD), and/or a Secure Digital (SD) card.

Further, embodiments may include a device (or standard waveform) database 118 that is configured to store the polling data received from the device 108. In one embodiment, the device database 118 may be configured to store the filtered RF signal received from the one or more RX antennas 112 of the device 108. The device database 118 may store the signal waveforms for the TX antenna 110 and the received signal waveforms for the RX antenna 112. The database may include the glucose readings with the corresponding signal waveform, received waveform and the TX antenna 110 and RX antenna 112 that were used. In some embodiments, the database may contain the TX antenna 110 and RX antennas 112 that were used, such as a first TX antenna 110 and a first RX antenna 112, a second TX antenna 110 and a second RX antenna 112, etc. as described in the sweep TX RX module 130, or the first TX antenna 110 and second RX antenna 112, the second TX antenna 110 and fourth RX antenna 112, etc. as described in the cross sweep TX RX module 132. Examples of implementation of the device network memory 116 may include, but are not limited to, Cloud storage, Cloud server, Random Access Memory (RAM), Read Only Memory (ROM), and/or a Secure Digital (SD) card.

Further, embodiments may include a processor 120 which may facilitate the operation of the device 108 with the device network to perform functions according to the instructions stored in the memory 116. In one embodiment, the processor 120 may include suitable logic, circuitry, interfaces, and/or code that may be configured to execute a set of instructions stored in the memory 116. The processor 120 may be configured to run the instructions obtained by the device base module 126 to perform polling. The processor 120 may be further configured to collect real-time signals from the one or more TX antennas 110 and the one or more RX antennas 112 and may store the real-time signals in the memory 116. In one embodiment, the real-time signals may be assigned as initial and updated radio frequency (RF) signals. Examples of the processor 120 may be an X86-based processor, a Reduced Instruction Set Computing (RISC) processor, an Application-Specific Integrated Circuit (ASIC) processor, a Complex Instruction Set Computing (CISC) processor, and/or other processors. The processor 120 may be a multicore microcontroller specifically designed to carry multiple operations based upon pre-defined algorithm patterns to achieve the desired result. Further, the processor 120 may take inputs from the device 108 and retain control by sending signals to different parts of the device 108. The processor 120 may consist of a Random Access Memory (RAM) that is used to store data and other results created when the processor 120 is at work. It can be noted that the data is stored temporarily for further processing, such as filtering, correlation, correction, and adjustment. Moreover, the processor 120 can carry out special tasks as programs that are pre-stored in the Read Only Memory (ROM). It can be noted that the special tasks carried out by the processor 120 indicate and apply certain actions which trigger specific responses.

Further, the communication module 122 of the device 108 may communicate with the device network via a cloud network. Examples of the communication module 122 may include, but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). In one embodiment, various devices may be configured to have a communication module integrated over circuitry arrangement to connect with the device network via various wired and wireless communication protocols, such as the cloud network. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), Zigbee, EDGE, infrared (IR), IEEE® 802.11, 802.16, cellular communication protocols, and/or Bluetooth® (BT) communication protocols.

Further, embodiments may include a battery 124 which may be disposed over the substrate to power hardware modules of the device 108. The device 108 may be configured with a charging port to recharge the battery 124. It can be noted that the charging of the battery 124 may be through wired or wireless means.

Further, embodiments may include a device base module 126 which may reside within the memory 116 or could be in a separate memory. The device base module 126 may provide all the instructions needed for device 108 or some or all instructions in device base module 126 may be executed in a cloud and network module (not shown). The communication module 122 can be used to communicate some or all of the device base module 126 instructions between the cloud or a network module and the device 108. The device base module 126 may be configured to store instructions for executing the computer program from the converted digital processor readable format of the ADC 114. The device base module 126 is configured to facilitate the operation of the processor 120, the memory 116, the one or more TX antennas 110, the one or more RX antennas 112, and the communication module 122. Further, the device base module 126 may be configured to create polling of the RF range radio frequency signals. It can be noted that the device base module 126 is configured to filter the RF range radio frequency signals received from the one or more RX antennas 112.

Further, embodiments may include a base operations module 128, as described in FIG. 8, which begins by initiating the system. The base operations module 128 loads the waveforms for transmission. The base operations module 128 saves the waveforms for transmission in the device database 118. The base operations module 128 continuously polls for a completion signal from the best sweep data extraction module 134. The base operations module 128 receives the completion signal from the best sweep data extraction module 134. The base operations module 128 sends a notification with the best results.

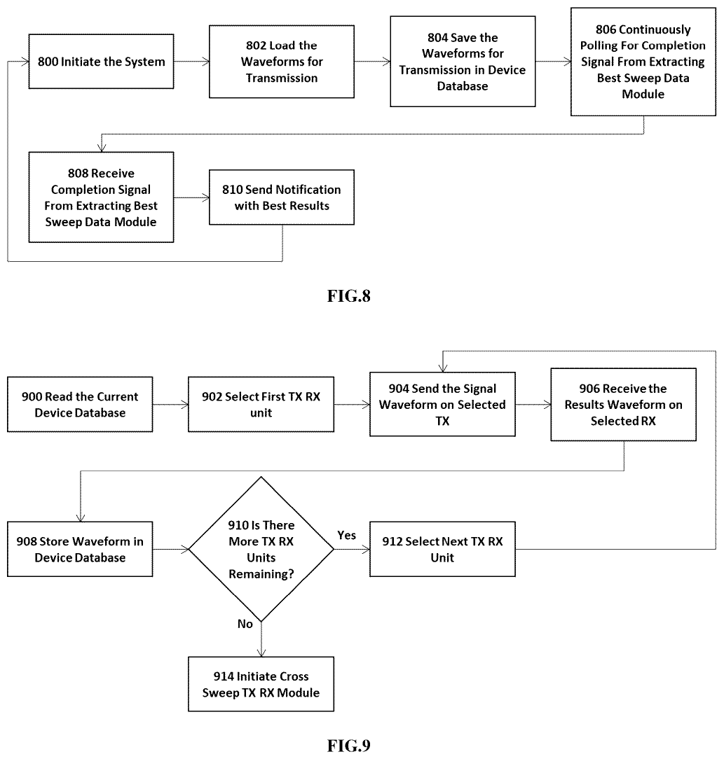

Further, embodiments may include a sweep TX RX module 130, as described in FIG. 9, which operates by reading the current device database 118. The sweep TX RX module 130 selects the first TX RX unit. The sweep TX RX module 130 sends the signal waveform on the selected TX. The sweep TX RX module 130 receives the results waveform from the selected RX antenna 112. The sweep TX RX module 130 stores the results waveform in the device database 118. The sweep TX RX module 130 determines if there are more TX RX units remaining. If it is determined that there are more TX RX units remaining the sweep TX RX module 130 selects the next TX RX unit and the process returns to sending the signal waveform on the selected TX. If it is determined that there are no more TX RX units remaining the sweep TX RX module 130 initiates the cross sweep TX RX module 132.

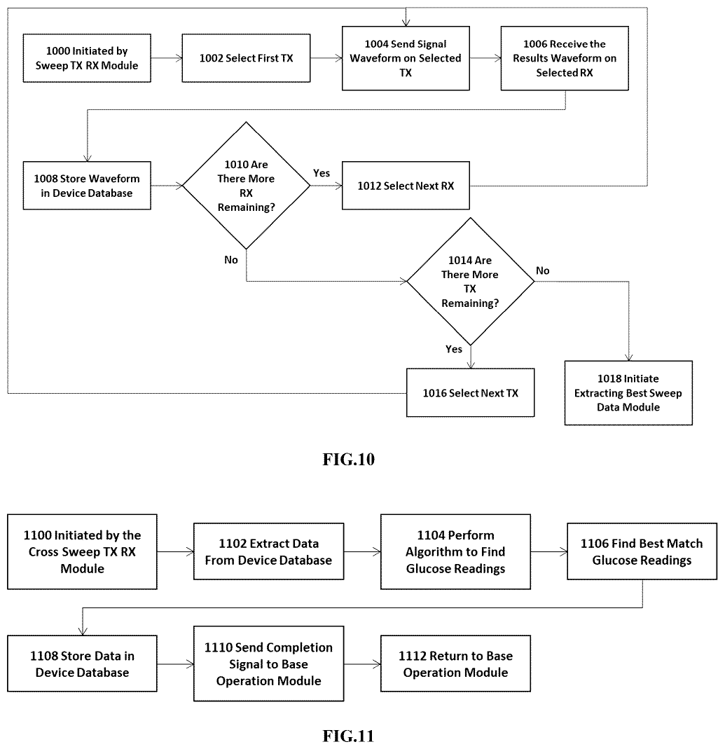

Further, embodiments may include a cross sweep TX RX module 132, as described in FIG. 10, which begins by being initiated by the sweep TX RX module 130. The cross sweep TX RX module 132 selects the first TX. The cross sweep TX RX module 132 sends the signal waveform on the selected TX. The cross sweep TX RX module 132 receives the results waveform on the selected RX. The cross sweep TX RX module 132 stores the results waveform in the device database 118. The cross sweep TX RX module 132 determines if there are more RX remaining. If it is determined that there are more RX remaining the cross sweep TX RX module 132 selects the next RX and the process returns to sending the signal waveform on the selected TX. If it is determined that there are no more RX remaining the cross sweep TX RX module 132 determines if there are more TX remaining. If it is determined that there are more TX remaining the cross sweep TX RX module 132 selects the next TX and the process returns to sending the signal waveform on the selected TX. If it is determined that there are no more TX remaining the cross sweep TX RX module 132 initiates the best sweep data extraction module 134.

Further, embodiments may include a best sweep data extraction module 134, as described in FIG. 11, which begins by being initiated by the cross sweep TX RX module 132. The best sweep data extraction module 134 extracts the data from the device database 118. The best sweep data extraction module 134 can perform an algorithm to find the glucose readings. The best sweep data extraction module 134 can find the best match glucose readings, for example as shown in FIG. 11 and described below. The best sweep data extraction module 134 stores the data in the device database 118. The best sweep data extraction module 134 sends completion signal to the base operations module 128. The best sweep data extraction module 134 returns to base operations module 128.

In some embodiments, the device base module 126 may utilize a motion module 136 that includes at least one sensor from the group of an accelerometer, a gyroscope, an inertial movement sensor, or other similar sensor. The motion module 136 may have its own processor or utilize the processor 120 to calculate the user's movement. Motion from the user will change the blood volume in a given portion of their body and the blood flow rate in their circulatory system. This may cause noise, artifacts, or other errors in the real-time signals received by the RX antennas 112. The motion module 136 may compare the calculated motion to a motion threshold stored in memory 116. For example, the motion threshold could be movement of more than two centimeters in one second. The motion threshold could be near zero to ensure the user is stationary when measuring to ensure the least noise in the RF signal data. When calculated motion levels exceed the motion threshold, the motion module 136 may flag the RF signals collected at the time stamp corresponding to the motion as potentially inaccurate. In some embodiments, the motion module 136 may compare RF signal data to motion data over time to improve the accuracy of the motion threshold. The motion module 136 may alert the user, such as with an audible beep or warning or a text message or alert to a connected mobile device. The alert would signal the user that they are moving too much to get an accurate measurement. The motion module 136 may update the device database 118 with the calculated motion of the user that corresponds with the received RF signal data. In this manner, the motion module 136 may be simplified to just collect motion data and allow the device base module 126 to determine if the amount of motion calculated exceeds a threshold that would indicate the received RF signal data is too noisy to be relied upon for a blood glucose measurement.

The device base module 126 may utilize a body temperature module 138 that includes at least one sensor from the group of a thermometer, a platinum resistance thermometer (PRT), a thermistor, a thermocouple, or another temperature sensor. The body temperature module 138 may have its own processor or utilize the processor 120 to calculate the temperature of the user or the user's environment. The user's body temperature, the environmental temperature, and the difference between the two will change the blood volume in a given part of their body and the blood flow rate in their circulatory system. Variations in temperature from the normal body temperature or room temperature may cause noise, artifacts, or other errors in the real-time signals received by the RX antennas 112. The body temperature module 138 may compare the measured temperature to a threshold temperature stored in memory 116. For example, the environmental temperature threshold may be set at zero degrees Celsius because low temperatures can cause a temporary narrowing of blood vessels which may increase the user's blood pressure. When the measured temperature exceeds the threshold, the body temperature module 138 may flag the RF signals collected at the time stamp corresponding to the temperature as potentially being inaccurate. In some embodiments, the body temperature module 138 may compare RF signal data to temperature data over time to improve the accuracy of the temperature threshold. The body temperature module 138 may alert the user, such as with an audible beep or warning or a text message or alert to a connected mobile device. The alert would signal to the user that their body temperature, or the environmental temperature is not conducive to getting an accurate measurement. The body temperature module 138 update the device database 118 with the measured user or environmental temperature that corresponds with the received RF signal data. In this manner, the body temperature module 138 may be simplified to just collect temperature data and allow the device base module 126 to determine if the temperature measure exceeds a threshold that would indicate the received RF signal data is too noisy to be relied upon for a blood glucose measurement.

The device base module 126 may utilize a body position module 140 that includes at least one sensor from the group of an accelerometer, a gyroscope, an inertial movement sensor, or another similar sensor. The body position module 140 may have its own processor or utilize the processor 120 to estimate the user's position. The user's body position may change the blood volume in a given part of their body and the blood flow rate in their circulatory system. This may cause noise, artifacts, or other errors in the real-time signals received by the RX antennas 112. The body position module 140 may compare the estimated position to a body position threshold stored in memory 116. For example, the monitoring device 102 may be on the user's wrist, and the body position threshold may be based on the relative position of the user's hand to their heart. When a user's hand is lower than their heart, their blood pressure will increase, with this effect being more pronounced the longer the position is maintained. Conversely, the higher a user holds their arm above their heart, the lower the blood pressure in their hand. The body position threshold may include some minimum amount of time the estimated body position occurs. When the estimated position exceeds the threshold, the body position module 140 may flag the RF signals collected at the time stamp corresponding to the body position as potentially being inaccurate. In some embodiments, the body position module 140 may compare RF signal data to motion data over time to improve the accuracy of the body position threshold. The body position data may also be used to estimate variations in parameters such as blood pressure that corresponds to the body position data to improve the accuracy of the measurements taken when the user is in that position. The body position module 140 may alert the user, such as with an audible beep or warning or a text message or alert to a connected mobile device. The alert would signal to the user that their body position is not conducive to getting an accurate measurement. The body position module 140 may update the device database 118 with the estimated body position data that corresponds with the received RF signal data. In this manner, the body position module 140 may be simplified to just collect temperature data and allow the device base module 126 to determine if the body position exceeded a threshold that would indicate the received RF signal data is too noisy to be relied upon for a blood glucose measurement.

The device base module 126 may utilize an ECG module 142 that includes at least one electrocardiogram sensor. The ECG module 142 may have its own processor or utilize the processor 120 to record the electrical signals that correspond with the user's heartbeat. The user's heartbeat will impact blood flow. Measuring the ECG data may allow the received RF data to be associated with peak and minimum cardiac output so as to create a pulse waveform allowing for the estimation of blood volume at a given point in the wave of ECG data. Variations in blood volume may cause noise, artifacts, or other errors in the real-time signals received by the RX antennas 112. The ECG module 142 may compare the measured cardiac data to a threshold stored in memory 116. For example, the threshold may be a pulse above 160 bpm, as the increased blood flow volume may cause too much noise in the received RF signal data to accurately measure the blood glucose. When the ECG data exceeds the threshold, the ECG module 142 may flag the RF signals collected at the time stamp corresponding to the ECG data as potentially being inaccurate. In some embodiments, the ECG module 142 may compare RF signal data to ECG data over time to improve the accuracy of the ECG data threshold or to improve the measurement of glucose at a given point in the cycle between peak and minimum cardiac output. The ECG module 142 may alert the user, such as with an audible beep or warning or a text message or alert to a connected mobile device. The alert would signal to the user that their heart rate is not conducive to getting an accurate measurement or requires additional medical intervention. The ECG module 142 may update the device database 118 with the measured ECG data that corresponds with the received RF signal data. In this manner, the ECG module 142 may be simplified to just collect ECG data and allow the device base module 126 to determine if the ECG data exceeded a threshold that would indicate the received RF signal data is too noisy to be relied upon for a blood glucose measurement.

The device base module 126 may utilize a circadian rhythm module 144 that includes at least one sensor measuring actigraphy, wrist temperature, light exposure, and heart rate. The circadian rhythm module 144 may have its own processor or utilize the processor 120 to calculate the user's circadian health. Blood pressure follows a circadian rhythm in that it increases upon waking in the morning and decreases during sleeping at night. People with poor circadian health will often have higher blood pressure. These variations in blood pressure can cause noise, artifacts, or other errors or inaccuracies in the real-time signals received by the RX antennas 112. The circadian rhythm module 144 may compare the circadian data to a threshold stored in memory 116. For example, the threshold may be less than 6 hours of sleep in the last 24 hours. When the observed circadian health data exceeds the threshold, the circadian rhythm module 144 may flag the RF signals collected at the time stamp corresponding to circadian health as potentially being inaccurate or needing an adjustment to account for the expected increase in the user's blood pressure. In some embodiments, the circadian rhythm module 144 may compare RF signal data to sleep data over time to improve the accuracy of the circadian rhythm thresholds. The circadian rhythm module 144 may alert the user, such as with an audible beep or warning, or a text message or alert to a connected mobile device. The alert would signal to the user that their recent sleep patterns are not conducive to getting an accurate measurement. The circadian rhythm module 144 may update the device database 118 with the measured circadian data that corresponds with the received RF signal data. In this manner, the circadian rhythm module 144 may be simplified to just collect circadian rhythm data and allow the device base module 126 to determine if the measure exceeded a threshold that would indicate the received RF signal data is too noisy to be relied upon for a blood glucose measurement, or if an alternative transfer function should be used to compensate for the detected circadian health.

The device base module 126 may include a received noise module 146 that includes at least one sensor measuring background signals such as RF signals, Wi-Fi, and other electromagnetic signals that could interfere with the signals received by the RX antennas 112. The received noise module 146 may have its own processor or utilize the processor 120 to calculate the level of background noise being received. Background noise may interfere with or cause noise, artifacts, or other errors or inaccuracies in the real-time signals received by the RX antennas 112. The received noise module 146 may compare the level and type of background noise to a threshold stored in memory 116. The threshold may be in terms of field strength (volts per meter and ampere per meter) or power density (watts per square meter). For example, the threshold may be RF radiation greater than 300 μW/m2. When the background noise data exceeds the threshold, the received noise module 146 may flag the RF signals collected at the time stamp corresponding to background noise levels as potentially being inaccurate. In some embodiments, the received noise module 146 may compare RF signal data to background noise over time to improve the accuracy of the noise thresholds. The received radiation module may alert the user, such as with an audible beep or warning, a text message, or an alert to a connected mobile device. The alert would signal to the user that the current level of background noise is not conducive to getting an accurate measurement. The received noise module 146 may update the device database 118 with the background noise data that corresponds with the received RF signal data. In this manner, the received noise module 146 may be simplified to just collect background noise data and allow the device base module 126 to determine if the measure exceeded a threshold that would indicate the received RF signal data is too noisy to be relied upon for a blood glucose measurement, or if an alternative transfer function should be used to compensate for the noise.

In embodiments, one or more of memory 116, the device database 118, the base operations module 128, the sweep TX RX module 130, cross sweep TX RX module 132, the best sweep data extraction module 134, the motion module 136, the body temperature module 138, the body position module 140, the ECG module 142, the circadian rhythm module 144, and/or the received noise module 146 can be provided on one or more separate devices, such as a cloud server, networked device, or the like. In such embodiments, the communication module 122 can be used to communicate with the cloud server or the networked device to access the memory 116, the device database 118, the base operations module 128, the sweep TX RX module 130, cross sweep TX RX module 132, the best sweep data extraction module 134, the motion module 136, the body temperature module 138, the body position module 140, the ECG module 142, the circadian rhythm module 144, and/or the received noise module 146 by way of any suitable network.

Further, an initial activated RF transmit signal TX can be sent and then there may be a measurement of the receive antenna RX signal, so these two signals around the same time window are associated. Generally, a series of Transmit TX signals in the RF Activated range are sent, one right after another, and associated signals from the RX receive antenna are stored. The series of activated RF Transmit TX signals TX1, TX2, TXn are different signals (frequencies and amplitudes). The are many possibilities of use cases as to how many and at what variabilities activated RF Transmit TX signals are used.

Prior to the real time use of the device 108, a systematic test can be done to build a set of activated RF transmit signals that have associated receive RX antenna 112 signals that can be analyzed, in which the analysis can be correlated to subjects that at the same time are taking ground truth blood samples for blood glucose. With a range of subjects having a range of blood glucose levels, this ground truth data can be trained against the set of activated RF transmit signals that have associated receive RX antenna signals so that the data saved in the device database 118 is robust enough to be used as ground truth RX antenna signals (with their associated glucose levels) to correlate to newly obtained real time RX antenna signals.

In order to start the process, the Transmit TX signals TX1, TX2, TXn are started from an initial TX1 signal and the receive antenna signal associated with this TX1 signal is obtained. The initial TX1 signal is then updated to say TX2 and the process repeats until all transmit TX signals are sent.

In an embodiment, the device 108 can be positioned at a wrist of the user. A typical wrist's depth is in a range of 40-60 mm (based on a wrist circumference in the range of 140-190 mm). The wrist can include the extensor carpi radialis brevis (ECRB), extensor carpi radialis longus (ECRL), extensor carpi ulnaris (ECU), extensor indicis proprius (EIP), extensor pollicis brevis (EPB), extensor pollicis longus (EPL), flexor carpi ulnaris (FCU), and flexor digitorum. The basilic vein may be roughly located in subcutaneous tissue under the skin.

It can be noted that the thickness of human skin in a wrist area is around 1-4 mm, and the thickness of the subcutaneous tissue may vary from 1-34 mm, although these thicknesses may vary based on many factors. It can be noted that the hand includes both capillaries having a diameter in the range of 5-10 microns, and the cephalic vein and the basilic vein having a diameter range of 1-4 mm. The capillaries, the cephalic vein, and the basilic vein may be approximately 1-9 mm below the skin of the hand. In one embodiment, the RF Activated range radio frequency signals may be particularly employed in pinpointing the position of a blood artery like the basilic vein and thereby monitoring the blood glucose level.

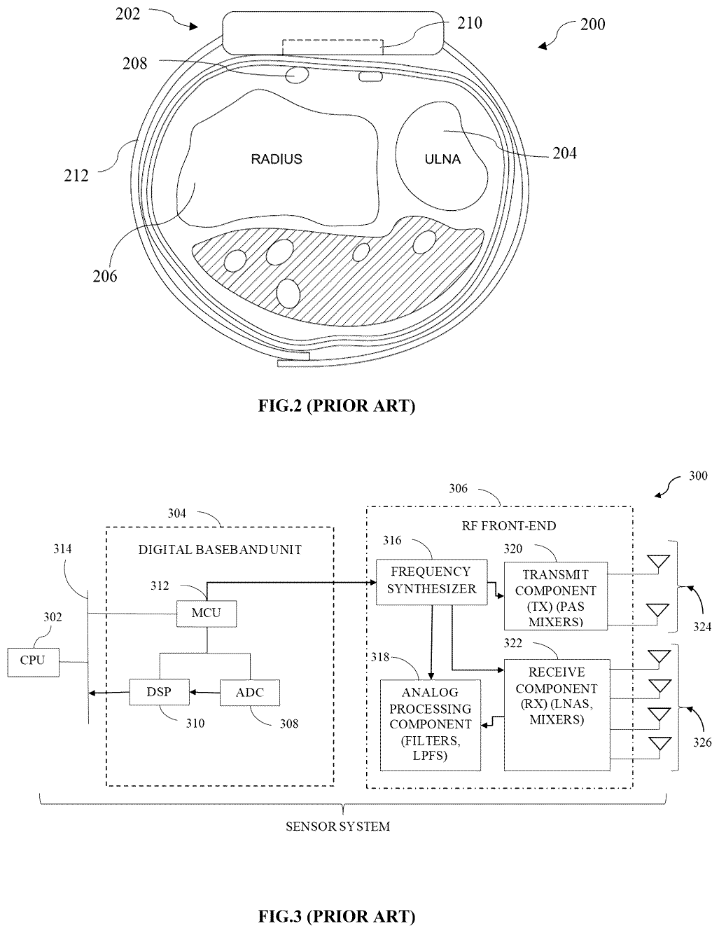

As shown in FIG. 2, the wrist 200 may be provided with a prior art device. In one example embodiment, the location of the device relative to the wrist 200 and relative to the basilic vein of the wrist 200 is depicted. The location of the device 202 relative to the anatomy of the wrist 200, including a radius bone 206, the ulna bone 204, and the basilic vein 208, is an important consideration in monitoring blood glucose levels using RF Activated range radio frequency signals. Further, a dashed line block (shown by 210) represents an approximate location of a sensor system (not shown) on the device. The sensor system is described in conjunction with FIG. 3. Further, the device may be provided with a strap 212 to tightly hold the device around the wrist 200 in a secured position. It can be noted that the strap 212 may be provided with multiple fastening means (not shown) to adjust the device around the wrist 200. The device may be configured to transmit the RF Activated range radio frequency signals towards the basilic vein and receive the RF Activated range radio frequency signals responded from blood components inside the basilic vein. It can be noted that a large quantity of RF Activated range radio frequency signals imparted underneath the skin of the wrist 200 may be responded from the radius bone 206 and/or the ulna bone 204 in the wrist 200 as well as from some dense tissue, such as tendons and ligaments, that are located between the skin and the bones at a posterior of the wrist 200.

FIG. 3 illustrates a functional block diagram of a prior art sensor system 300. The sensor system 300 may comprise a central processing unit (CPU) 302, a digital baseband unit 304, and a radio frequency (RF) front end 306. Further, the digital baseband unit 304 may comprise an analog-to-digital converter (ADC) 308, a digital signal processor (DSP) 310, and a microcontroller unit (MCU) 312. In one embodiment, the digital baseband unit 304 may include some other configurations, including some other combination of elements. The digital baseband unit 304 may be connected to the CPU 302 using bus connectors 314.

Further, the RF front-end 306 may comprise a frequency synthesizer 316, an analog processing component 318, a transmit (TX) component 320, and a receive (RX) component 322. Further, the TX component 320 may include PAS elements. The PAS elements correspond to power, amplifiers, and mixers. The RX component 322 may include LNAS elements. The LNAS elements correspond to low noise amplifiers (LNAs), variable gain amplifiers (VGAs), and mixers. The frequency synthesizer 316 may include elements to generate electrical signals at frequencies used by the TX component 320 and the RX components 322. In one embodiment, the frequency synthesizer 316 may include elements such as a crystal oscillator, a phase-locked loop (PLL), a frequency multiplier, and a combination thereof. The analog processing component 318 may include elements such as mixers and filters. In one embodiment, the filters may include low-pass filters (LPFs). In one embodiment, the frequency synthesizer 316, the analog processing component 318, the TX component 320, and the RX component 322 of the RF front end 306 may be implemented in hardware as electronic circuits that are fabricated on the same semiconductor substrate.

Further, the TX component 320 may comprise at least two TX antennas 324, and the RX component 322 may comprise at least four RX antennas 326. In one embodiment, the sensor system 300 may be provided with multiple TX antennas and RX antennas in a ratio of 1:2.

Further, at least two TX antennas 324 and the at least four RX antennas 326 may be configured to transmit and receive RF Activated-range radio frequency signals. In one embodiment, the sensor system 300, including the CPU 302, the digital baseband unit 304, and the RF front end 306 of the monitoring device 202, may be integrated into various configurations according to the size and shape of the monitoring device 202. For example, some configurations of components of the monitoring device 202 are fabricated on a semiconductor substrate and/or included in a packaged IC device or a combination of packaged IC devices. The TX antennas 324 are formed on the semiconductor substrate or included in one or more packaged IC devices such that the TX antennas 324 are in a planar two-dimensional (2D) array relative to one another. The RX antennas 326 are formed on the semiconductor substrate or included in one or more packaged IC devices such that the RX antennas 326 are in a planar 2D array relative to one another. In one embodiment, the device 108 is designed to transmit and receive RF Activated radio frequency signals at a pre-defined frequency. In one embodiment, the pre-defined frequency ranges between 122-126 GHz RF Activated range radio frequency signals.

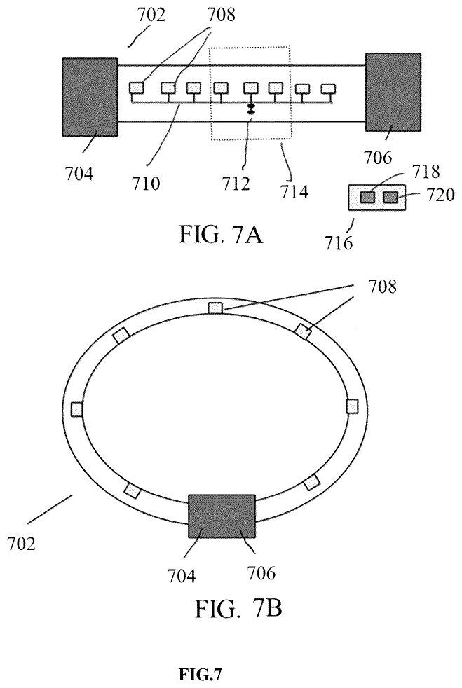

When the TX antennas 110 transmit millimeter range radio waves, the electromagnetic energy may have a three-dimensional (3D) illumination pattern. The 3D illumination pattern can be generated by distributing the plurality of TX antennas 110 in a three-dimensional array as illustrated in FIG. 7A and FIG. 7B. Given the three-dimensional pattern as illustrated in FIGS. 7A and 7B, the plurality of TX antennas 110 can illuminate an area that has a maximum coverage around the wrist. The 3D arrays of TX antennas 110 and RX antennas 112 is an array where the elements are distributed in all three dimensions of length, width, and height. For example, the 3D array may allow the TX antenna 110 and RX antenna 112 to provide coverage in multiple directions, including up and down, which allows the TX antenna 110 to send and the RX antenna 112 to receive signals from multiple angles. The 3D array may be designed to have TX antennas 110 and RX antennas 112 to be spatially dispersed throughout the device 108 to facilitate diversity reception, which improves the reliability and quality of the signal by reducing fading, interference, and multipath effects. The 3d array of the TX antennas 110 and RX antennas 112 allows the ability to capture weaker signals or transmit stronger signals.

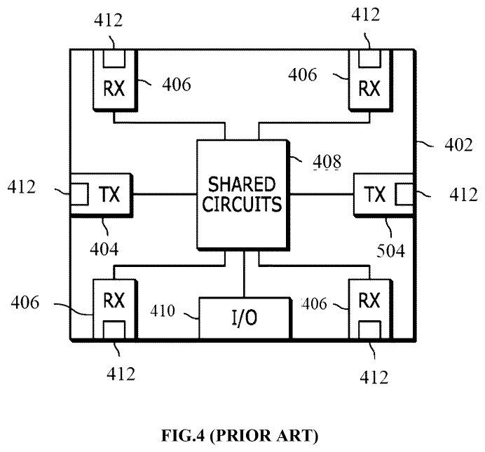

FIG. 4 illustrates an example of a prior art physical layout of circuit components on a semiconductor substrate. In the embodiment of FIG. 4, the die 402 includes two TX components 404, four RX components 406, shared circuits 408, and an input/output interface (I/O) 410. In the example of FIG. 4, each TX component 404 includes channel-specific circuits (not shown) such as amplifiers, each RX component 406 includes channel-specific circuits (not shown) such as mixers, filters, and LNAs, and the shared circuits include, for example, a voltage control oscillator (VCO), a local oscillator (LO), frequency synthesizers, PLLs, BPFs, divider(s), mixers, ADCs, buffers, digital logic, a DSP, CPU, or some combination thereof that may be utilized in conjunction with the channel-specific TX and RX components. As shown in FIG. 4, the transmit and receive components 404 and 406 each include an interface 412 (such as a conductive pad) that provides an electrical interface between the circuits on the die and a corresponding antenna.

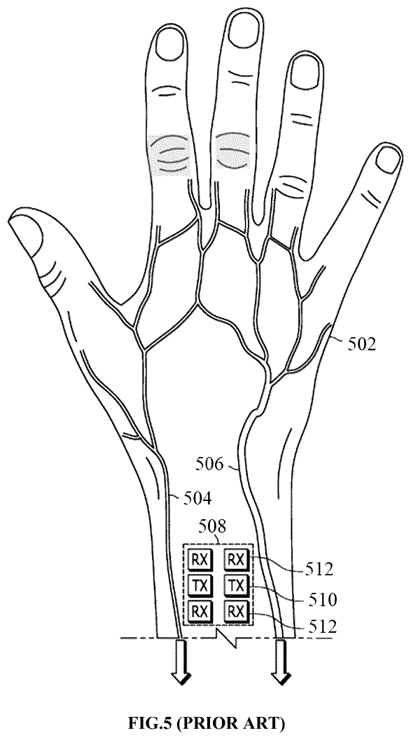

FIG. 5 illustrates a prior art IC device 508 overlaid on the hand/wrist 502. The IC device is oriented with regard to the basilic and cephalic veins 504 and 506 such that the two TX antennas 510 are configured transverse to the basilic and cephalic veins and the RX antennas 512 are on both sides of the TX antennas 510. That is, the two TX antennas are distributed transversely relative to the orientation (e.g., the linear direction) of the vessel or vessels that will be monitored, such as the basilic and cephalic veins. For example, in a transverse configuration, a straight line that passes through the two TX antennas would be transverse to the vessel or vessels that will be monitored, such as the basilic and cephalic veins. In an embodiment in which the wearable device is worn on the wrist, the transverse configuration of the TX antennas is such that a line passing through both of the TX antennas is approximately orthogonal to the wrist and approximately orthogonal to the orientation of the vessel or vessels that will be monitored, such as the basilic and cephalic veins. For example, a line passing through both of the TX antennas and the orientation of the vessel or vessels that will be monitored, such as the basilic and cephalic veins, may be without about 20 degrees F.rom orthogonal.

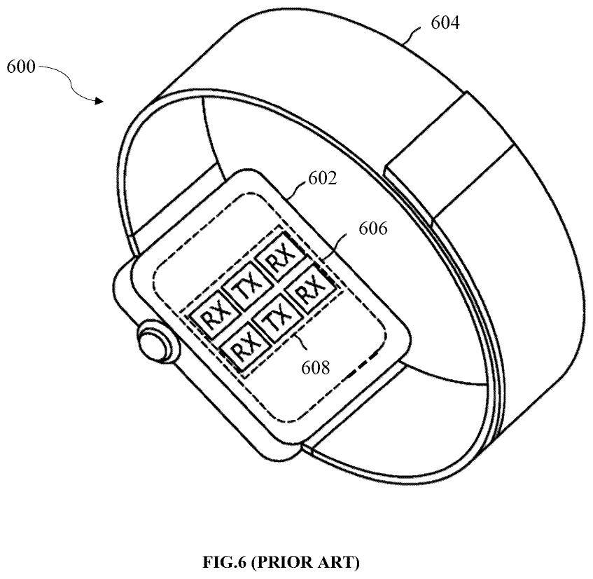

FIG. 6 illustrates a prior art IC device 608 overlaid on the back of the smartwatch 600. The two TX antennas are configured such that when the smartwatch is worn on the wrist, the two TX antennas are transverse to veins such as the basilic and cephalic veins that run parallel to the length of the arm and wrist. In a case in which the two TX antennas are configured transverse to veins such as the basilic and cephalic veins of a person wearing the smartwatch 600. The two TX antennas are spatially separated from each other such that both of the TX antennas are visible from the side view. When the TX antennas transmit RF Activated range radio waves, the electromagnetic energy may have a 3D illumination pattern.

It has been established that the amount of glucose in the blood (blood glucose level) affects the response of RF Activated range radio waves. However, when RF Activated range radio waves are applied to the human body (e.g., at or near the skin surface), electromagnetic energy is responded from many objects including the skin itself, fibrous tissue such as muscle and tendons, and bones. In order to effectively monitor a health parameter such as the blood glucose level of a person, electrical signals that correspond to electromagnetic energy that is responded from blood (e.g., from the blood in a vein) should be isolated from electrical signals that correspond to electromagnetic energy that is responded from other objects such as the skin itself, fibrous tissue, and bone, as well as from electrical signals that correspond to electromagnetic energy that is emitted directly from the TX antennas (referred to herein as electromagnetic energy leakage or simply as “leakage”) and received by an antenna without passing through the skin of the person.

Various techniques that can be implemented alone or in combination to isolate electrical signals that correspond to reflections from blood from other electrical signals that correspond to other reflections (such as reflections from bone and/or fibrous tissue such as muscle and tendons) and/or signals that correspond to leakage are described below. Such techniques relate to and/or involve, for example, transmission characteristics, beamforming, Doppler effect processing, leakage mitigation, and antenna design.

FIG. 7A-7B illustrate an embodiment of a device integrated in a wrist band, with FIG. 7A displaying a top view of the wrist band 702 and FIG. 7B displaying a side view of the wrist band. The wrist band 702 may include a plurality of TX RX pairs 708, a left buckle 704, a right buckle 706, a bus 710, connectors 712, a housing unit 714, a TX RX antenna pair 716, a TX antenna 718, and a RX antenna 720. The wrist band 702 may be a smart device, such as smart watch, a bracelet, a band, strap, etc. that a user may place on their wrist. The wrist band 702 may be included in a smart watch, such as an embodiment of device 108 shown in FIG. 1 and described above. The left buckle 704 may connect to the right buckle 706 to secure the wrist band 702 to the user's wrists. The buckles 704 and 706 may connect through a clasp, magnets, Velcro, etc. The plurality of TX RX pairs 708 may each be an IC device that contains a TX antenna 718 and a RX antenna 720 in which the one or more TX antennas 718 may be configured to transmit the RF Activated range radio frequency signals at a pre-defined frequency. In one embodiment, the pre-defined frequency may correspond to a range suitable for the human body. The plurality of TX RX pairs 708 may be positioned in a series around the wrist band 702 to provide a three-dimensional array when the wrist band 702 is secured over the wrist. When the wrist band 702 is bent around the wrist of the subject, the TX RX pairs distributed through the wrist band 702 take positions that are not in plane with one another. The TX antennas 718 of the TX RX pairs 708 form a three-dimensional (3D), non-planar array relative to one another. The RX antennas 720 of the TX RX pairs 708 form a 3D, non-planar array relative to one another. In some embodiments, there may be a plurality TX RX pairs 708 in a series and in multiple rows around the wrist band 702 to provide a three-dimensional array when the wrist band 702 is secured. For example, the one or more TX antennas 718 can transmit signals within the RF Activated range or a smaller portion thereof, for example radio frequency signals at a range of 120-126 GHz. Successively, the one or more RX antennas 720 may be configured to receive the responded portion of the RF Activated range radio frequency signals. In one embodiment, the RF Activated range radio frequency signals may be transmitted to the user's skin, and electromagnetic energy may be responded from many parts such as fibrous tissue, muscle, tendons, bones, and the skin. It can be noted that effective monitoring of the blood glucose level is facilitated by an electrical response of blood molecules, such as pancreatic endocrine hormones, against the transmitted RF Activated range radio frequency signals. It will be apparent to a skilled person that the pancreatic endocrine hormones such as insulin and glucagon are responsible for maintaining sugar or glucose level. Further, the electromagnetic energy responded from the blood molecules may be received by the one or more RX antennas 720. A controller may be used to communicate, via the bus 710, with the devices that are contained within the wrist band 702 through physical connections such as cables or printed circuits. The controller transmits a variety of control signals to components and devices to transmit control signals to the controller using the bus 710. One of the main objectives of a bus is to minimize the lines that are needed for communication. The bus 710 may be bidirectional and assists the controller in synchronizing control signals to internal devices and external components. It is comprised of interrupt lines, byte enable lines, read/write signals and status lines. The connectors 712 may be an electro-mechanical device used to create an electrical connection between the plurality of TX RX pair 708. The connectors 712 may receive power, data signals, informational instructions, etc. from the connector to power and control the individual TX RX pair 708. The housing 714 may be a protective layer over the TX RX pair 708 to protect the components of the wrist band 702, which may be made of plastic, glass, silicon, etc. The TX RX antenna pair 716 may be an IC device that contains a TX antenna 718 and a RX antenna 720 in which the one or more TX antennas 718 may be configured to transmit the RF Activated range radio frequency signals at a pre-defined frequency. In one embodiment, the pre-defined frequency may correspond to a range suitable for the human body. For example, the one or more TX antennas 718 transmit signals within the RF Activated range radio frequency signals at a range of 120-126 GHz. Successively, one or more RX antennas 720 may be configured to receive the responded portion of the RF Activated range radio frequency signals. In one embodiment, the RF Activated range radio frequency signals may be transmitted to the user's skin, and electromagnetic energy may be responded from many parts such as fibrous tissue, muscle, tendons, bones, and the skin. It can be noted that effective monitoring of the blood glucose level is facilitated by an electrical response of blood molecules, such as pancreatic endocrine hormones, against the transmitted RF Activated range radio frequency signals. It will be apparent to a skilled person that the pancreatic endocrine hormones such as insulin and glucagon are responsible for maintaining sugar or glucose level. Further, the electromagnetic energy responded from the blood molecules may be received by the one or more RX antennas 720. When the TX antennas 718 transmit millimeter range radio waves, the electromagnetic energy may have a three-dimensional (3D) illumination pattern as illustrated in FIG. 7A and FIG. 7B. Given the three-dimensional pattern as illustrated in FIGS. 7A and 7B, the plurality of TX antennas 718 illuminate an area that has a maximum coverage around the wrist. In some embodiments, the three-dimensional array may be a 3d array which may be a multi-dimensional array or an array of arrays, to increase the illumination pattern provided by the device 108.

FIG. 8 illustrates operation of the base operations module 128. The process begins with the base operations module 128 initiating, at step 800, the system. For example, the base operations module 128 begins the process of loading and storing the waveforms that will be sent by the plurality of TX antennas 110 and storing the received result waveforms from the plurality of RX antennas 112. The base operations module 128 loads, at step 802, the waveforms for transmission. For example, the base operations module 128 loads the waveforms for transmission by the one or more TX antennas 110 for ultimate detection by the one or more RX antennas 112. The base operations module 128 saves, at step 804, the waveforms for transmission in the device database 118. For example, the base operations module 128 stores the waveforms that will be transmitted by the TX antennas 110 in the device database 118. In one embodiment, the pre-defined frequency may correspond to a range suitable for the human body. For example, the one or more TX antennas 110 transmit signals within the RF Activated range radio frequency signals at a range of 120-126 GHz. The base operations module 128 continuously polls, at step 806, for a completion signal from the best sweep data extraction module 134. For example, the base operations module 128 is continuously polling to receive a completion signal from the best sweep data extraction module 134 to be signaled that the process is complete. The base operations module 128 receives, at step 808, the completion signal from the best sweep data extraction module 134. For example, the base operations module 128 receives a completion signal from the best sweep data extraction module 134 to be signaled that the process is complete. The base operations module 128 sends, at step 810, a notification with the best results. For example, the base operations module 128 may send the notification to the user of the best results, such as through the device 108, by sending the best results to a device network to be notified through a computer or smart device, such as a mobile device, etc.

FIG. 9 illustrates operation of the sweep TX RX module 130. The process begins with the sweep TX RX module 130 reading, at step 900, the device database 118. For example, the sweep TX RX module 130 reads the device database 118 to extract the waveform that is to be sent by the TX antenna 110. In some embodiments, the device database 118 may have different waveforms that will be sent by different TX antennas 110. The sweep TX RX module 130 selects, at step 902, the first TX RX unit. For example, the sweep TX RX module 130 selects the first TX RX unit, such as the first TX RX unit in a device 108 that contains a plurality of TX RX units. The sweep TX RX module 130 sends, at step 904, the signal waveform on the selected TX. For example, the sweep TX RX module 130 sends the signal waveform extracted from the device database 118 on the selected TX antenna 110. For example, the pre-defined frequency may correspond to a range suitable for the human body. For example, the one or more TX antennas 110 transmit signals within the RF Activated range radio frequency signals at a range of 120-126 GHz. The sweep TX RX module 130 receives, at step 906, the results waveform from the selected RX antenna. For example, the sweep TX RX module 130 receives the results waveform from the selected RX antenna 112, such as the RX antenna 112 that is in the same unit as the TX antenna 110 that sent the signal waveform. The one or more RX antennas 112 may be configured to receive the responded portion of the RF Activated range radio frequency signals. In one embodiment, the RF Activated range radio frequency signals may be transmitted to the user's skin, and electromagnetic energy may be responded from many parts such as fibrous tissue, muscle, tendons, bones, and the skin. It can be noted that effective monitoring of the blood glucose level is facilitated by an electrical response of blood molecules, such as pancreatic endocrine hormones, against the transmitted RF Activated range radio frequency signals. It will be apparent to a skilled person that the pancreatic endocrine hormones such as insulin and glucagon are responsible for maintaining sugar or glucose level. Further, the electromagnetic energy responded from the blood molecules may be received by the one or more RX antennas 112. The sweep TX RX module 130 stores, at step 908, the results waveform in the device database 118. For example, the sweep TX RX module 130 stores the results waveform in the device database 118. In some embodiments, the TX antenna 110 that sent the signal waveform, the signal waveform that was sent, the TX RX unit that was used, etc. may also be stored in the device database 118. The sweep TX RX module 130 determines, at step 910, if there are more TX RX units remaining. For example, the device 108 may contain a plurality of TX RX units and the sweep TX RX module 130 may select each TX antenna 110 and RX antenna 112 individually to send a signal waveform with the TX antenna 110 and receive the results waveform from the RX antenna 112 paired with the TX antenna 110 that sent the signal waveform until all of the TX RX units have sent and received waveforms. If it is determined that there are more TX RX units remaining the sweep TX RX module 130 selects, at step 912, the next TX RX unit and the process returns to sending the signal waveform on the selected TX antenna 110. For example, the sweep TX RX module 130 continues selecting the next TX RX unit until all of the TX RX units have sent and received a results waveform. If it is determined that there are no more TX RX units remaining the sweep TX RX module 130 initiates, at step 914, the cross sweep TX RX module 132.

FIG. 10 illustrates operation of the cross sweep TX RX module 132. The process begins with the cross sweep TX RX module 132 being initiated, at step 1000, by the sweep TX RX module 130. For example, the cross sweep TX RX module 132 is initiated by the sweep TX RX module 130. In some embodiments, the sweep TX RX module 130 may send the waveforms to be sent by the plurality of TX antennas 110. In some embodiments, the cross sweep TX RX module 132 may read the device database 118 to extract the waveform that is to be sent by the TX antenna 110. In some embodiments, the device database 118 may have different waveforms that will be sent by different TX antennas 110. The cross sweep TX RX module 132 selects, at step 1002, the first TX. For example, the cross sweep TX RX module 132 selects the first TX antenna 110, such as the first TX antenna 110 in a device 108 that contains a plurality of TX RX units. The cross sweep TX RX module 132 sends, at step 1004, the signal waveform on the selected TX. For example, the cross sweep TX RX module 132 sends the signal waveform extracted from the device database 118 on the selected TX antenna 110. For example, the pre-defined frequency may correspond to a range suitable for the human body. For example, the one or more TX antennas 110 transmit signals within the Activated range radio frequency signals at a range of 120-126 GHz. The cross sweep TX RX module 132 receives, at step 1006, the results waveform on the selected RX. For example, the cross sweep TX RX module 132 receives the results waveform from the selected RX antenna 112, such as the second TX RX antenna 112 that is in a different unit than the TX antenna 110 that sent the signal waveform. The one or more RX antennas 112 may be configured to receive the responded portion of the RF Activated range radio frequency signals.

For example, if there are 10 TX RX 708 pairs on the wrist band 702, the TX antenna 110 of the first pair is used to send out an RF signal. Then, the RX antenna 112 of the first pair 708 is used to receive and store the return signal. Then, the next RX antenna 112 for the second pair 708 is used to receive and store this signal. This is continued until all RX antennas 112 of all 708 pairs are used to collect all the RF signals. In this way, one TX antenna 110 would be used to collect RF signals from all the different RX antenna 112 of TX RX pairs 708. This process is continued so that the next TX RX pair is used, using the TX antenna 110 of the second pair TX RX 708. After the data is received on the RX antenna 112 of the second TX RX pair, the next RX antenna 112 of the next TX RX pair 708 is polled and that RF signal is collected. The process continues until all the TX antennas 110 and RX antennas 112 are used. It should be noted that it may be likely that the RX antenna 112 may have the strongest signal when the TX antenna 110 in the same TX RX pair is used (due to proximity and the attenuation of RF waves in the human body). But, the RX antenna 112 data signals might be quite different for an RX antenna 112 proximate to the initiated TX antenna 110 of a TX RAX pair 708, versus the RX antenna 112 in a TX RX pair 708 across the wrist. But it is likely the entire process could be repeated with various strengths of signals sent to TX antennas 110 so that the signal to RX antennas 112 across the wrist can be improved. In this way, more received RF data is received to analyze that takes into account a more 360 degrees around the wrist effects, which could help correct mis information looking only for received antenna RX waves in proximate TX RX pairs 708.

In one embodiment, the RF Activated range radio frequency signals may be transmitted into the user's skin, and electromagnetic energy may be responded from many parts such as fibrous tissue, muscle, tendons, bones, and the skin. It can be noted that effective monitoring of the blood glucose level is facilitated by an electrical response of blood molecules, such as pancreatic endocrine hormones, against the transmitted RF Activated range radio frequency signals. It will be apparent to a skilled person that the pancreatic endocrine hormones such as insulin and glucagon are responsible for maintaining sugar or glucose level. Further, the electromagnetic energy responded from the blood molecules may be received by the one or more RX antennas 112. The cross sweep TX RX module 132 stores, at step 1008, the results waveform in the device database 118. For example, the cross sweep TX RX module 132 stores the results waveform in the device database 118. In some embodiments, the TX antenna 110 that sent the signal waveform, the signal waveform that was sent, the RX unit that received the results waveform, etc. may also be stored in the device database 118. The cross sweep TX RX module 132 determines, at step 1010, if there are more RX antenna 112 remaining. For example, the device 108 may contain a plurality of TX RX units and the cross sweep TX RX module 132 may select the first TX antenna 110 to send a signal waveform and receive the results waveform from the second RX antenna 112, then the first TX antenna 110 would send another waveform and receive the results waveform from the third RX antenna 112, then the first TX antenna 110 would send another waveform and receive the results waveform from the fourth RX antenna 112, until all of the RX antennas 112 have received a result waveform that was sent by the first TX antenna 110. If it is determined that there are more RX remaining the cross sweep TX RX module 132 selects, at step 1012, the next RX antenna and the process returns to sending the signal waveform on the selected TX antenna. For example, the device 108 may contain a plurality of TX RX units and the cross sweep TX RX module 132 may select the first TX antenna 110 to send a signal waveform and receive the results waveform from the second RX antenna 112, then the first TX RX antenna 110 would send another waveform and receive the results waveform from the third RX antenna 112, then the first TX antenna 110 would send another waveform and receive the results waveform from the fourth RX antenna 112, until all of the RX antennas 112 have received a result waveform that was sent by the first TX antenna 110. If it is determined that there are no more RX antennas remaining the cross sweep TX RX module 132 determines, at step 1014, if there are more TX antennas remaining. For example, once all of the RX antennas 112 have received a result waveform that was sent by the selected TX antenna 110 the process is repeated with the next TX antenna 110. For example, the cross sweep TX RX module 132 may select the second TX antenna 110 to send a signal waveform and receive the results waveform from the third RX antenna 112, then the second TX antenna 110 would send another waveform and receive the results waveform from the fourth RX antenna 112, then the second TX antenna 110 would send another waveform and receive the results waveform from the fifth RX antenna 112, until every combination of the plurality of TX antennas 110 have sent a signal waveform that has been received by the plurality of the RX antennas 112. If it is determined that there are more TX remaining the cross sweep TX RX module 132 selects, at step 1016, the next TX antenna 110 and the process returns to sending the signal waveform on the selected TX antenna 110. For example, once all of the RX antennas 112 have received a result waveform that was sent by the selected TX antenna 110 the process is repeated with the next TX antenna 110. For example, the cross sweep TX RX module 132 may select the second TX antenna 110 to send a signal waveform and receive the results waveform from the third RX antenna 112, then the second TX antenna 110 would send another waveform and receive the results waveform from the fourth RX antenna 112, then the second TX antenna 110 would send another waveform and receive the results waveform from the fifth RX antenna 112, until every combination of the plurality of TX antennas 110 have sent a signal waveform that has been received by the plurality of the RX antennas 112. If it is determined that there are no more TX remaining the cross sweep TX RX module 132 initiates, at step 1018, the best sweep data extraction module 134.

FIG. 11 illustrates operation of the best sweep data extraction module 134. The process begins with the best sweep data extraction module 134 being initiated, at step 1100, by the cross sweep TX RX module 132. The best sweep data extraction module 134 extracts, at step 1102, the data from the device database 118. For example, the device database 118 may be configured to store the filtered RF signal received from the one or more RX antennas 112 of the device 108. The device database 118 may store the signal waveforms for the TX antenna 110 and the received signal waveforms for the RX antenna 112. The database may include the glucose readings with the corresponding signal waveform, received waveform and the TX antenna 110 and RX antenna 112 that were used. In some embodiments, the database may contain the TX antenna 110 and RX antennas 112 that were used, such as the first TX antenna 110 and first RX antenna 112, the second TX antenna 110 and second RX antenna 112, etc. as described in the sweep TX RX module 130, or the first TX antenna 110 and second RX antenna 112, the second TX antenna 110 and fourth RX antenna 112, etc. as described in the cross sweep TX RX module 132. The best sweep data extraction module 134 performs, at step 1104, an algorithm to the find the glucose readings. For example, the device database 118 may contain a series of waveforms sent by a TX antenna 110, result waveforms received by a RX antenna 112, a corresponding pulse waveform related to the result waveform and a glucose reading related to the pulse waveform. The algorithm compares each of the sent waveforms and result waveforms to the device database to identify a sent and result waveform that is previously stored and that closely matches the sent and received waveforms and then extracts the corresponding pulse waveform and glucose reading from the previously stored data entry and stores the data with the sent and result waveform. This process is completed for all of the sent and result waveforms that were performed in the sweep TX RX module 130 and cross sweep TX RX module 132 until all of the data entries have a corresponding pulse waveform and glucose reading. In some embodiments, the algorithms performed may those disclosed in US 20220192494A1, which is herein incorporated by reference. The best sweep data extraction module 134 finds, at step 1106, the best match glucose readings. For example, the algorithm may determine a percentage indicating how closely the sent and result waveforms mirror particular previously stored waveforms to determine a best match. For example, if previous ground truth blood sample glucose readings corresponding to, for example, 75 mg/dl are found to be associated to a particular waveform stored in the database, then the correlated waveforms matched would relate to the stored glucose readings of the prestored ground truth glucose data. In one embodiment, machine learning is used to relate new received waveforms to the existing waveforms (and associated ground truth glucose readings). In another embodiment, correlations algorithms are used to relate new received waveforms to the existing waveforms (and associated ground truth glucose readings).

To further the example, if the sent waveform from the third TX antenna 110 and the result waveform from the fourth RX antenna 112 matches a previously stored data entry at 98% that may be the highest percentage of all the waveforms sent and received and compared to the device database 118 which would result in that being the best match, resulting in the glucose reading for that data entry being used to notify the user. The best sweep data extraction module 134 stores, at step 1108, the data in the device database 118. For example, the best sweep data extraction module 134 stores the data in the device database 118 such as the TX antenna 110 used, the RX antenna 112 used, the waveform signal sent, the waveform signal received, the related pulse waveform and the glucose reading for all the waveforms sent and received during the processes described in the sweep TX RX module 130 and cross sweep TX RX module 132. The best sweep data extraction module 134 sends, at step 1110, completion signal to the base operations module 128. For example, once each of the sent and received waveforms have had the algorithm performed then the best sweep data extraction module 134 sends a completion signal to the base operations module 128 to signal that the process is complete. In some embodiments, the best sweep data extraction module 134 may send the best match to the base operations module 128 or may send the glucose readings to be used to notify the user. The best sweep data extraction module 134 returns, at step 1112, to base operations module 128.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A health monitoring system, comprising:
a wristband;
antenna pairs distributed along the wristband, each antenna pair includes a transmit antenna configured to transmit radio frequency (RF) detection signals into a person wearing the wristband and a receive antenna configured to detect RF return signals resulting from transmitting the RF detection signals into the person; wherein when the wristband is secured to the person, the antenna pairs are disposed in a non-planar, three-dimensional array;
a controller connected to the antenna pairs and controlling the antenna pairs to:
transmit first RF detection signals having a frequency into the person using a first transmit antenna of one of the antenna pairs and generate first collected data corresponding to detected first RF return signals from the person in response to the transmitted first RF detection signals, wherein the detected first RF return signals are received by a plurality of the receive antennas of the antenna pairs;
(b) repeat (a) at a plurality of additional frequencies to generate additional collected data; and
determine a value that corresponds to a glucose level in the person based on the first collected data and the additional collected data.

2. The health monitoring system of claim 1, wherein the controller is further configured to control the antenna pairs to:
(c) transmit second RF detection signals having a frequency into the person using a second transmit antenna of one of the antenna pairs and generate second collected data corresponding to detected second RF return signals from the person in response to the transmitted second RF detection signals, wherein the detected second RF return signals are received by a plurality of the receive antennas of the antenna pairs;
(d) repeat (c) at a plurality of additional frequencies to generate second additional collected data; and
determine the value that corresponds to the glucose level in the person based on the first collected data, the additional collected data, the second collected data and the second additional collected data.

3. The health monitoring system of claim 1, wherein the controller is part of a device that is physically separate from the wristband.

4. A method for monitoring a glucose level of a person, the method comprising:
(a) transmitting first radio waves having a frequency into the person using a first transmit antenna and generating first collected data corresponding to received first radio waves from the person in response to the transmitted first radio waves, wherein the received first radio waves are received by a plurality of receive antennas disposed in a non-planar, three-dimensional array;
(b) repeating (a) at a plurality of additional frequencies to generate additional collected data; and
determining a value that corresponds to a glucose level in the person based on the first collected data and the additional collected data.

5. The method of claim 4, comprising:
(c) transmitting second radio waves having a frequency into the person using a second transmit antenna and generating second collected data corresponding to received second radio waves from the person in response to the transmitted second radio waves, wherein the received second radio waves are received by the plurality of receive antennas;
(d) repeating (c) at a plurality of additional frequencies to generate second additional collected data; and
determining the value that corresponds to the glucose level in the person based on the first collected data, the additional collected data, the second collected data and the second additional collected data.

6. The method of claim 5, wherein the plurality of receive antennas are disposed in a wristband.

7. The method of claim 6, wherein the first transmit antenna and the second transmit antenna are disposed in the wristband.
